(19) United States
(12) Patent Application Publication
(10) Pub. No.: US 2013/0096368 A1
(43) Pub. Date: Apr. 18, 2013

(54) UTERINE SOUND AND MOTION SIMULATION DEVICE

(71) Applicant: Edmond M. Devroey, Chula Vista, CA (US)

(72) Inventor: Edmond M. Devroey, Chula Vista, CA (US)

(21) Appl. No.: 13/639,071

(22) PCT Filed: Nov. 18, 2010
(86) PCT No.: PCT/US10/57157

§ 371 Date: Oct. 2, 2012

Related U.S. Application Data
(60) Provisional application No. 61/281,594, filed on Nov. 19, 2009.

(51) Int. Cl. A61M 21/02

(52) U.S. Cl. CPC A61M 21/02
USPC 600/28; 29/896.2

(57) ABSTRACT

The present invention is directed to a Uterine Sound and Motion Simulation Device either incorporated Into an incubator or portable that a baby will lay on consisting of an outer enclosure that folds in half inclosing an inner sound and motion actuation unit housing one or more gaii, or body motion units using bladders or pillows actuated by the means of an external air, liquid or gas source, controlled by the means of plurality of internal or external valves activated through a computer control unit. A control program in the control unit will produce the movement ‘motion experienced by the baby while in the womb uterus by the means of a bladder or pillow or both, that is inflated and deflated in a manner that mimics the movement/motion of the baby while in the womb/uterus. One or more speakers will be located within the activation unit and will operate through the same control unit producing the sounds heard in the uterus including the mother's voice.

### FIELD OF THE INVENTION

The present application relates to the field of devices used in the care of newborn or prematurely born babies in the neonatal intensive care units of hospitals or in the home. More particularly, as used in the neonatal intensive care units of hospitals the present invention will be useful in helping to alleviate the consequences of solitary confinement of premature babies in their incubator on later development and behavior and as used in the home environment the present invention will be useful in helping to alleviate the consequences of endless crying on the development of the newborn.

### BACKGROUND OF THE INVENTION

In the last ten years, Boston psychologist Heidelise Als and others have researched and developed ways of making nurseries and neonatal care more “baby friendly.” This approach is known as individualized developmental care (or formally, Neonatal Individualized Developmental Care and Assessment Program, NIDCAP). Therapies that have brought about sharp decreases in neonatal mortality have not brought about similar decreases in neurodevelopmental morbidity for preterm infants. While other staff training programs for developmentally supportive care exists, it is the Newborn Developmental Care and Assessment Program (NIDCAP) that has been the methodology used in randomly controlled trials that demonstrate positive medical and neurobehavioral outcomes for preterm infants receiving developmentally supportive care.

Neonatal intensive care units, NICUs that care for all out-born or transported infants face unique challenges in balancing developmentally supportive nursing care with the high-technological medical needs of the most critically ill and extremely premature infants. Yet it is these most fragile infants who can most benefit from the positive impact the NIDCAP program can offer. Yet, there are no published studies documenting the impact of the NIDCAP program on neurobehavioral outcomes of these transported preterm infants exclusively.

There are many different kinds of technology in the NICU, including different types of infant ventilators, monitors, and supportive devices, such as infusion pumps, oxygen hoods, and incubators. Although this equipment has become more responsive to the needs of the infants, there is still much the scientific and medical communities can learn about the use of technology to support neonates. The many, tubes and sensors used for monitoring the baby's condition, blood sampling and artificial feeding makes some babies scarcely visible beneath the technology.

In the uterus, weight is reduced because fetuses float in the amniotic fluid and loses in weight the weight of the displaced fluid. In uteri the temperature is constant. A fetus never experiences temperature changes and ignores the discomfort that goes with it. In uteri, there is no vision. Researchers believe that a fetus may detect a reddish glow if the abdominal wall is thin enough and directly exposed to brilliant sunlight. Otherwise, darkness is the rule. Sounds are plentiful and the audition develops as early as the fourth month of pregnancy. The sounds from the external world are perceived, filtered by the abdominal and the uterus wall and are transmitted deadened through the amniotic fluid. Other than sounds of the external world, a fetus also hears sounds from its immediate surroundings. The sounds from the mother, her heartbeat, her respiration and her voice; the sounds of the blood flow through the placenta and the main arteries, and above all the loud noise produced by the maternal bowel moving liquids and gasses.

One may say that the uterus is a very loud place. The fetus experiences constant motion. He perceives the back and forth swings of his mother's respiration, the regularity of blood flow impulses, as well as the random movement of the mother. The mobility of the fetus is reduced by the proximity of the uterus wall. This restriction increases toward the term of the pregnancy. In uteri, available space is scarce. There is no smell in uteri, and taste along with touch is monotonous. Because of almost constant food supply, hunger perception is limited. After birth, the gravity exerts its full strength. At birth light is a very new perception. The perception of the external sounds is totally different when received through an air filled ear canal. The maternal sounds, except the voice, totally disappear after birth, as well as the motion of the respiratory swings, the impulses of the blood flow and the motion due to maternal movement.

After birth, the mobility is greater but restricted by the full weight perception and by clothes. After birth, the space becomes unlimited. The smell perception starts as soon as the nose is aerated. Taste becomes variable and intermittent, and hunger may become voracious.

In an incubator, the temperature (and the humidity) is maintained constant. Food is provided by ways appropriated to the age of the premature infant. An incubator frill of “mechanical” and “technical” sounds, while sounds and motion specific to the uterine environment are missing. The device we present is a substitute for the sounds and motion that are part of the natural environment in uterus.

Numerous innovations for the Uterine Sound and Motion Simulation Device have been provided in the prior art that are described as follows. Even though these innovations may be suitable for the specific individual purposes to which they address, they differ from the present design as hereinafter contrasted. The following is a summary of those prior art patents most relevant to this application at hand, as well as a description outlining the difference between the features of the Uterine Sound and Motion Simulation Device and the prior art.

U.S. Pat. No. 7,346,949 of Lori Kamrin-Balfour describes an infant soothing and sleep aid includes a fabric mat. A pair of removable and adjustable barriers are provided, one on either side, that act as a cradling mechanism to make the infant feel safe and secure in the large crib. The barriers include a soothing device such as a vibration mechanism and white noise mechanism in order to soothe the baby in the form of restricted movement, pleasing sounds, and motion.

This patent describes an infant soothing and sleep aid device including a fabric mat using a vibrating mechanism and a noise mechanism but does not exactly mimic the motion and sounds incurred in the womb by the baby.

U.S. Pat. No. 7,636,594 of Richard L. Watson describes an infant warming and sensor mat for keeping a baby warm and monitoring the baby's hemoglobin oxygen saturation level (SaO.sub.2) and heart rate. The mat preferably comprises a bottom layer of compressible padding material, a sensor layer comprising a plurality of light sources and light detectors, a cover layer having multiple openings, and an air layer between the sensor layer and cover layer. Warm air supplied to the air layer escapes through the openings in the cover to warm the baby. The light sources shine light of an appropriate wavelength onto the baby through the openings in the cover, and the light detectors sense the amount of light reflected back from the baby, which is indicative of SaO.sub.2 level. Heart rate is also determined from the resulting periodic signal.

This patent describes an infant warming and sensor mat for keeping a baby warm and monitoring the baby's hemoglobin oxygen saturation level but also does not exactly mimic the motion and sounds incurred in the womb by the baby.

U.S. Pat. No. 5,865,771 of Toshio Shuto, et al. describes an electromechanical vibration transducers are embedded in a pad, and a mat on which a newborn baby is placed on. A graphic equalizer is arranged to correct the frequency characteristics of a sound signal generated by a sound signal generator to characteristics close to the original frequency characteristics even if sounds are reflected to interfere with each other in an incubator. Comfortable vibrations can be applied to the newborn baby. In addition, sounds having frequency characteristics close to the original frequency characteristics can be played for the newborn baby. The newborn baby therefore can feel at ease, and at the same time bedsores, night terrors, and the like can be prevented. Maintenance cost is also low.

This patent describes electromechanical vibration transducers that are embedded in a pad creating frequency characteristics of a sound signal creating comfortable vibrations along with sounds relieving bedsores and night terrors. This patent also does not exactly mimic the motion and sounds incurred in the womb by the baby.

None of these previous efforts, however, provides the benefits attendant with the Uterine Sound and Motion Simulation Device. The present design achieves its intended purposes, objects and advantages over the prior art devices through a new, useful and unobvious combination of method steps and component elements, with the use of a minimum number of functioning parts, at a reasonable cost to manufacture, and by employing readily available materials. Other products presently on the market for such use are considered possibly dangerous nursery products, and do not achieve the desired result of preventing endless crying by the newborn, and the consequences of endless crying which may occur later in life.

In this respect, before explaining at least one embodiment of the Uterine Sound and Motion Simulation Device in detail it is to be understood that the design is not limited in its application to the details of construction and to the arrangement, of the components set forth in the following description or illustrated in the drawings. The Uterine Sound and Motion Simulation Device is capable of other embodiments and of being practiced and carried out in various ways. In addition, it is to be understood that the phraseology and terminology employed herein are for the purpose of description and should not be regarded as limiting. As such, those skilled in the art will appreciate that the conception, upon which this disclosure is based, may readily be utilized as a basis for designing of other structures, methods and systems for carrying, out the several purposes of the present design. It is important, therefore, that the claims be regarded as including such equivalent construction insofar as they do not depart from the spirit and scope of the present application.

### SUMMARY OF THE INVENTION

The principal advantage of the Uterine Sound and Motion Simulation Devices is to improve the care of newborn or prematurely born babies in the neonatal intensive care units of hospitals or in the home.

Another advantage of the Uterine Sound. and Motion Simulation Device is to give the newborn or prematurely born babies some of the experiences that were felt in the womb.

Another advantage of the Uterine Sound and Motion Simulation Device is to make use of the advanced computer technology to create a more lifelike experience similar to that felt in the womb.

Another advantage of the Uterine Sound and Motion Simulation Device is it will have bladders or pillows or both, for respiration and gait movements.

Another advantage of the Uterine Sound and Motion Simulation Device is being able to create a motion similar to that the baby feels in the womb.

Another advantage of the. Uterine Sound and Motion Simulation Device is being able to reproduce the exact body sounds heard by the baby in the womb.

Another advantage of the Uterine Sound and Motion Simulation Device is being able to reproduce the sounds of the mother's voice that the baby hears in the womb.

Yet another advantage of the Uterine Sound and Motion Simulation Device is being able to adjust, by the means of computer controlled operations, the timing and intensity of the sound and motion delivered by the device.

And still another advantage of the Uterine Sound and Motion Simulation Device is that it can be easily incorporated into an existing incubator in a hospital or be used as a portable device in the home.

A further advantage is low cost of manufacturing and sales of the Uterine Sound and Motion Simulation Device compared to the common medical devices used in hospitals.

These together with other advantages of the Uterine Sound and Motion Simulation Device, along with the various features of novelty, which characterize the design, are pointed out with particularity in the claims annexed to and forming a part of this disclosure. For a better understanding of the Uterine Sound and Motion Simulation Device, its operating advantages and the specific objects attained by its uses, reference should be made to the accompanying drawings and descriptive matter in which there are illustrated preferred embodiments of the Uterine Sound and Motion Simulation Device. There has thus been outlined, rather broadly, the more important features of the design in order that the detailed description thereof that follows may be better understood, and in order that the present contribution to the art may be better appreciated. There are additional features of the Uterine Sound and Motion Simulation Device that will be described hereinafter and which will form the subject matter of the claims appended hereto.

The Uterine Sound and Motion Simulation Device will consist of an outer enclosure made from a washable soft supple fabric that will fold in half with a hook-loop fastening material on three edges. One or more square hook-loop fastening squares are located on each side join together through matching orifices in the inner sound and motion activation unit to affix it firmly into position when the outer enclosure is folded together. For hygienic purposes the outer enclosure is washable.

The inner sound and motion activation unit will consist of a durable supple material container, housing one or more gait, or body motion units using bladders or pillows activated by the means of an external air, liquid or gas source, controlled by the means of plurality of internal or external valves activated through a computer control unit. These can operate by the means of compress material in tank form or by the means of a compressor or pump system. A control program in the computer control unit will produce the movement/motion experienced by the baby while in the womb/uterus by the means of a bladder or pillow or both, that is inflated and deflated in a manner that mimics the movement/motion of the baby while in the womb/uterus.

One or more speakers will be located within the activation unit and operated through the same control unit. The sound tracks can be independently recorded to facilitate fine tuning for the particular application. The sounds and movement may be customized to be exactly those of the mother or by the use of a standard program that is devised to match a mother with a particular set of physical characteristics or conditions. There will be one or more square orifices matching the square hook-loop fastening squares in the outer enclosure, holding the device firmly in position.

This novel device solves the problem of not having sound and movement that mimic's the environment that is experienced by the fetus in the uterus by providing a device that furnishes both sound and motion and will mimic the intra-uterine conditions. The baby will lay on top of the folded outer enclosure with the sound and motion unit in it. A unique support member to be used under the sealed outer enclosure has been devised that has a gentle head to feet slope of five degrees for the comfort of the baby and lateral body supports on the sides for the baby's safety. The support member is made of foam covered with a washable fabric. Such devices would contribute substantially in reducing the stress of the postnatal period and facilitate adaptation to the newborn's new life environment. The support is optional and intended for use with Example Models 1 and 2 described in greater detail below.

A unique sound and motion recorder device will be used to record the body sounds and movements of the mother including the mothers voice throughout a normal twenty four hour period. Sound from the heartbeat, respiratory tract, bowel activity and maternal voices are recorded as perceived by the fetus while in the uterus. Motion would be provided to mimic the intra-uterine motion by alternately swelling and relaxing one or more pillow(s) or bladder(s).

The preferred embodiment of the Uterine Sound and Motion Simulation Device will be built directly into the incubator when manufactured or installed as an accessory to existing incubator systems. The sound and motion sequence will be produced in either a digital or analog format and may be tailored to a specific set of conditions. The system may be used to stand alone, or integrated into the incubator electronics. The control unit will contain computer programs for display on screen and easy monitoring and adjustment of the controller functions. It will contain separate sound tracts for heartbeat, respiration, mother's voice, bowel and environmental noise. Each tract has an on/off switch and is programmable in time and duration. It will have software and buttons for respiration and gait movement adjustments allowing time, interval and intensity control. It will also come with the recorder to be carried by the mother during the twenty four hour recording period recording movements, sounds, inclusive heart beat, bowel, voice and environment. It will additionally incorporate the unique support member to be used under the sealed outer enclosure that has a gentle head to feet slope of five degrees for the comfort of the baby and two lateral body supports for the baby's safety.

The first alternate embodiment will be manufactured as a portable unit to be used in the hospital or in the home. The sound and motion sequence will be produced in either a digital or analog format and may be tailored to a specific set of conditions. The computer control unit will contain separate sound tracts for heartbeat, respiration, mother's voice, bowel and environmental noise. Each tract has an on/off switch and is programmed in time and duration. It will have software and buttons for respiration and gait movement adjustments allowing time, interval and intensity control. It will also incorporate the unique support member to be used under the sealed outer enclosure that has a gentle head to feet slope of five degrees for the comfort of the baby and two lateral body supports for the baby's safety.

The second alternate embodiment will be a less expensive version being a portable unit to be used in the hospital or in the home with the sound sequence only produced in either a digital or analog format and may be tailored to a specific set of conditions. The control unit will contain separate sound tracts for heartbeat, respiration, mother's voice, bowel and environmental noise. It will also optionally incorporate the unique support member to be used under the sealed outer enclosure that has a gentle head to feet slope of five degrees for the comfort of the baby and two lateral body supports for the baby's safety.

The foregoing has outlined rather broadly the more pertinent and important features of the present Uterine Sound and Motion Simulation Device in order that the detailed description of the application that follows may be better understood so that the present contribution to the art may be more fully appreciated. Additional features of the design will be described, hereinafter which for the subject of the claims of this disclosure. It should be appreciated by those skilled in the art that the conception and the disclosed specific embodiment may be readily utilized as a basis for modifying or designing other structures and methods for carrying out the same purposes of the present design. It should also be realized by those skilled in the art that such equivalent constructions and methods do not depart from the spirit and scope of this application as set forth in the appended claims.

### BRIEF DESCRIPTION OF THE DRAWINGS

The accompanying drawings, which are incorporated in and form a part of this specification, illustrate embodiments of the Uterine Sound and Motion Simulation Device and together with the description, serve to explain the principles of this application.

FIG. 1 depicts a perspective view of the Uterine Sound and Motion Simulation Device incorporated within an incubator.

FIG. 2A depicts a recording device having four microphones connected via four wires; this recorder will also be capable of recording respiration and gait movements.

FIG. 2B depicts a view of a pregnant woman or a new mother wearing the recording device shown in FIG. 2A, as the recordings may occur before or after birth of the new baby.

FIG. 3A depicts a perspective view of the components of the first alternate embodiment of the Uterine Sound and Motion Simulation Device.

FIG. 3B depicts a schematic diagram of the control unit, speakers, air/vacuum source and valves which function to produce sound and motion simulation, under the control of the control unit.

FIG. 4 depicts a perspective view of the Uterine Sound and Motion Simulation Device with a baby in phantom laying on it.

FIG. 5 depicts a perspective view of the components of the second alternate embodiment of the Uterine Sound and Motion Simulation Device.

FIG. 6 depicts a chart showing a baby's intrauterine and after birth perceptions.

FIG. 7 depicts algorithm A depicting the air flow control for the respiration bag with fixed values.

FIG. 8 depicts algorithm B depicting the air flow control for the gait bag.

FIG. 9 depicts algorithm A depicting the variable air flow control for the respiration bag.

FIG. 10 depicts algorithm B depicting the constant air flow for the gait bags.

FIG. 11 depicts algorithm C depicting the sound track control.

FIG. 12 depicts timing chart for respiration and gait motion.

FIG. 13 depicts a chart depicting the pump connections for the gait and respiration bags.

FIG. 14 depicts a chart depicting the respiration bags sequence.

FIG. 15 depicts a chart depicting the gait bags cycles.

For a filler understanding of the nature and advantages of the Uterine Sound and Motion Simulation Device, reference should be had to the following detailed description taken in conjunction with the accompanying drawings which are incorporated in and form a part of this specification, illustrate embodiments of the design and together with the description, serve to explain the principles of this application.

### DETAILED DESCRIPTION OF THE PREFERRED EMBODIMENTS

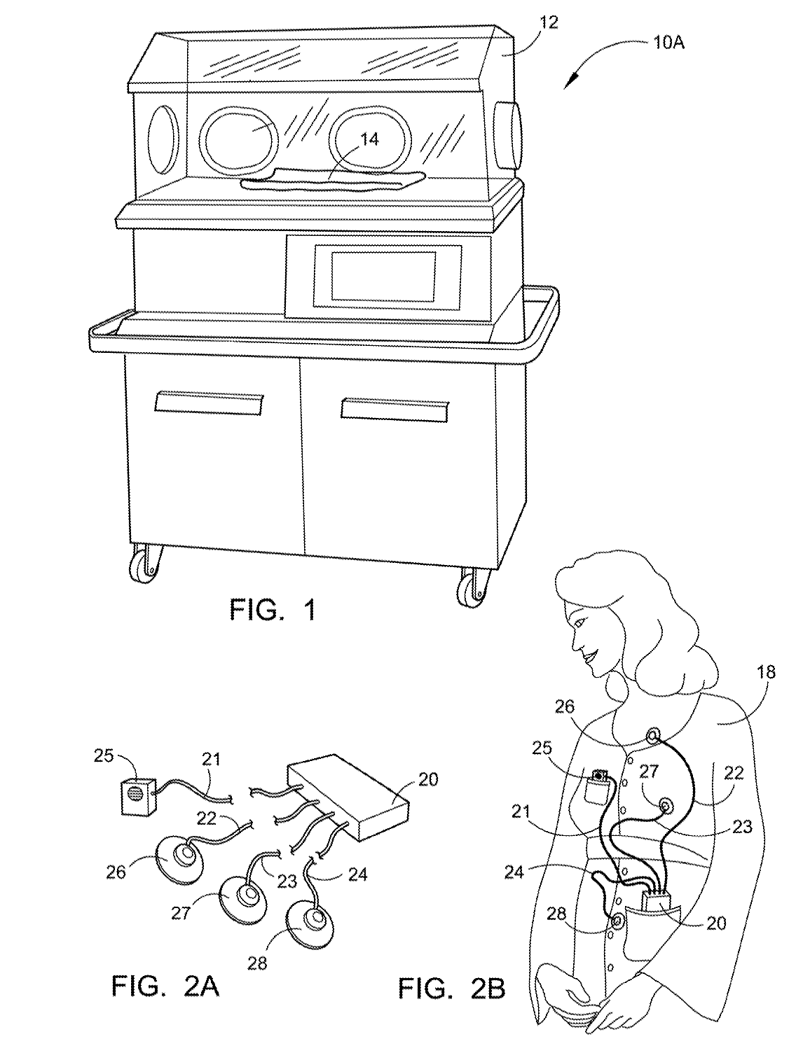

Referring now to the drawings, wherein similar pans of the Uterine Sound and Motion Simulation Devices 10A, 10B and 10C are identified by like reference numerals, there is seen in FIG. 1 a perspective view of the Uterine Sound and Motion Simulation Device 10A incorporated within an incubator 12 illustrating the outer enclosure 14 of the Uterine Sound and Motion Simulation Device 10A.

FIG. 2A depicts a view of the recording device 20. It is comprised of four connecting recorder wires 21, 22, 23 and 24 leading to tow recording microphones 25, 26, 27 and 28, Fewer or more microphones may be employed. The recording device 20 also acts as a motion recorder as well as a sound recording device. FIG. 2B depicts a view of a pregnant woman or new mother 18 wearing the recording device 20, either before or after giving birth, in the area of the upper chest (wire 22, microphone 26), the chest near the heart (wire 23, microphone 27), and to the abdomen (wire 24, microphone 28). Also, an external sound recording device (wire 21, external microphone 25) is worn on the outside of the clothing to record ambient sounds outside the new mother's body.

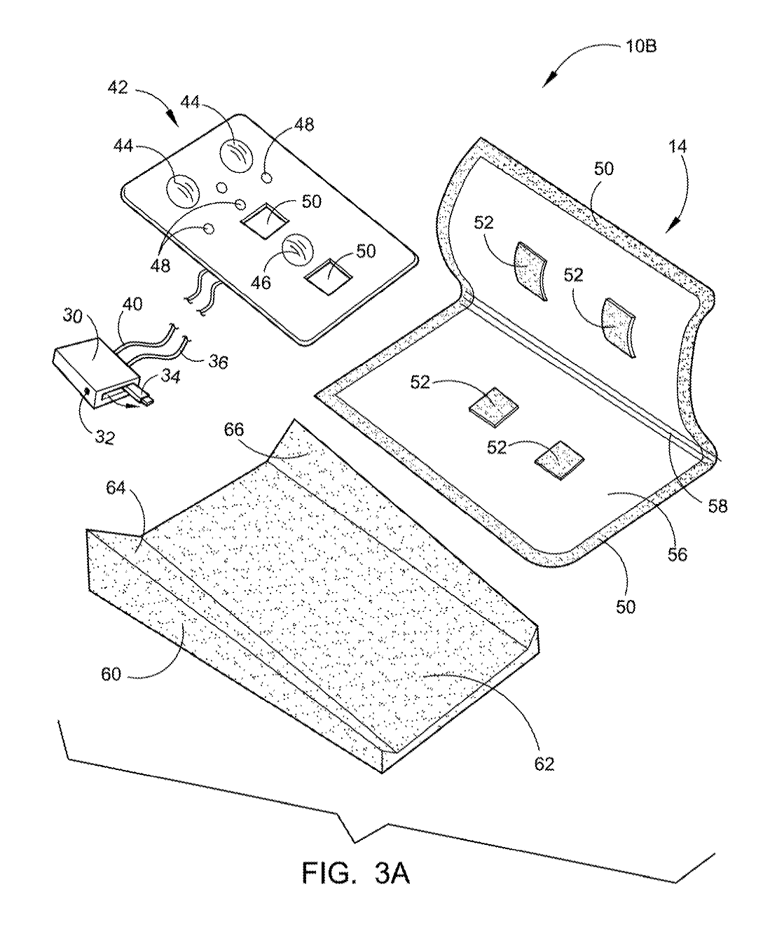

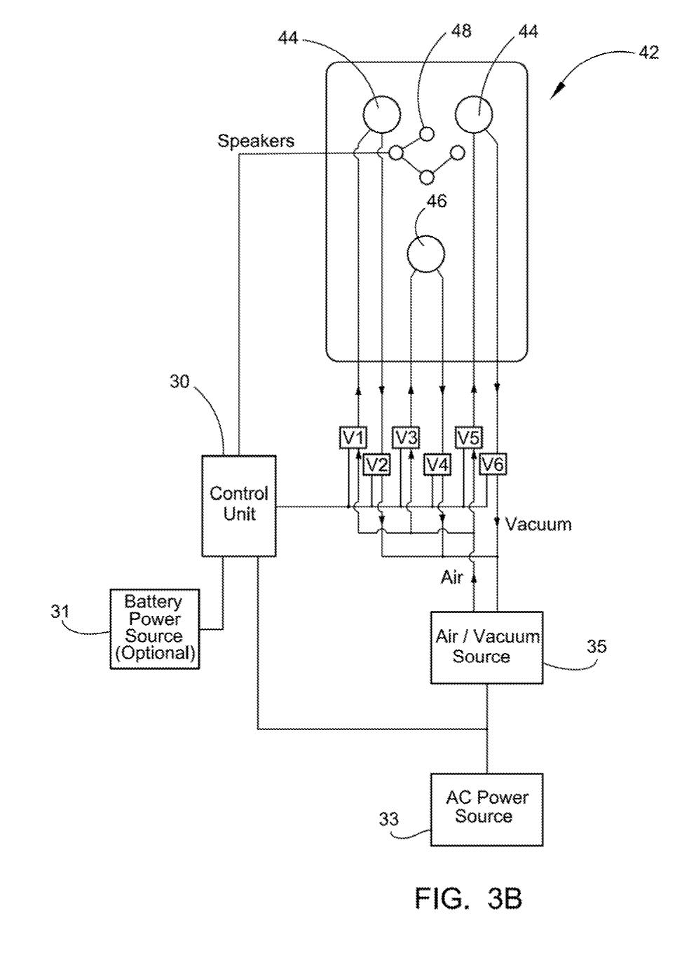

FIG. 3A depicts a perspective view of the components of the first alternate embodiment of the Uterine Sound and Motion Simulation Device 10B consisting of a control unit 30 having an AC power source 32 and a computer connection means 34, here shown as a standard USB port connection. Using the air, liquid or gas control line 36 and body motion and respiration line 40 the control unit 30 communicates and controls the function of the inner sound and motion activation unit 42. The Uterine Sound and Motion Simulation Devices 10 contains within it both the gait or body motion bladders or pillows 44 and the respiration bladders or pillow 46. One or more speakers 48 are conveniently placed in the area of the baby's head. One or more locating square orifices 50 are centrally located to match the square locating hook-loop fastening squares 52 centrally located on either side within the outer enclosure 14 holding the inner sound and motion activation unit 42 firmly in position.

FIG. 3B depicts a schematic of a control unit, speakers, air/vacuum source and valves which function to produce sound and motion simulation, under the control of the control unit. The control unit 30 may be battery 31 operated or may be powered by the means of an AC power source 33. The control unit 30 is in communication with and controls the air/vacuum source 35. Leading from the air/vacuum source 35 are a plurality of air lines and vacuum lines. The air/vacuum source is controlled directly by the control unit to send air (in the direction of the arrows shown), or pull a vacuum (in the direction of the arrows shown) to or from the gait or body motion bladders or pillows 44 and the respiration bladders or pillow 46 to simulate gait or respiration motions. The control unit 30 controls the opening and closing of plurality of valves, here six valves are shown as V1, V2, V3, V4, V5 and V6, or by means of a plurality of internal or external valves leading to the inner sound and motion activation unit 42. Fewer or more valves may be employed.

The control unit 30 is also in communication with the speakers 48 located within the inner sound and notion activation unit 42 and sound to the speakers is controlled by the control unit 30. The varying sound track may be programmed by computer, and played back in a variety of controlled sequences.

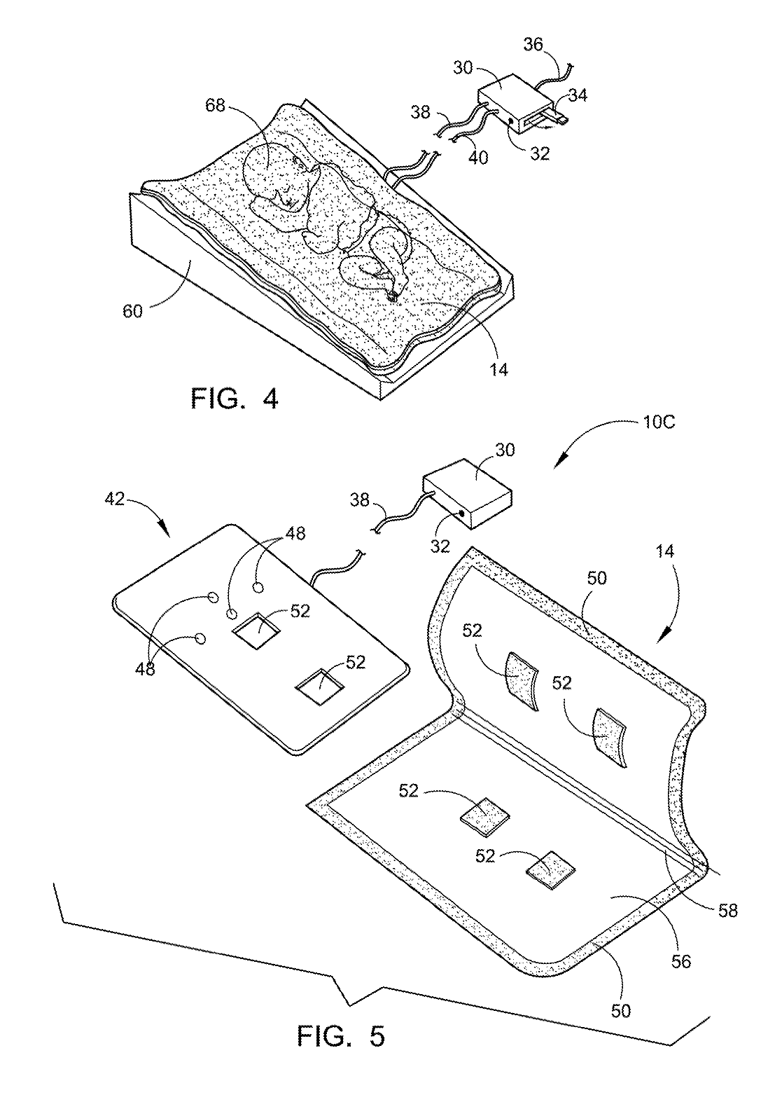

The outer enclosure 14 is made from a washable soft supple fabric that will fold in half with a hook-loop fastening edge material 54 on three edges. One or more square hook-loop fastening squares 52 are located on each side of the inner surface 56 to join together through matching orifices 50 in the inner sound and motion activation unit 42. A break in the hook-loop fastening material 54 will facilitate the clearance for the recording output line 38 and the body motion and respiration lines 40 (see FIG. 4 below).

The optional support member 60 to be used under the sealed outer enclosure 14 has a central flat surface 62 with a gentle head to feet slope of five degrees for the comfort of the baby and two lateral body supports two outside edges 64 and 66 for the baby's safety.

FIG. 4 depicts a perspective view of the Uterine Sound and Motion Simulation Device 10B with a baby 68 in phantom laying on it. Here, the control unit shown also has the capability of connecting to a computer for programming movement and sound. A swing out USB port 34, or similar device connector, will facilitate the connection. A break in the hook-loop fastening material 54 will facilitate the clearance for the recording output line 38 and the body motion and respiration lines 40.

FIG. 5 depicts a perspective view of the components of the second alternate embodiment of the Uterine Sound and Motion Simulation Device 10C consisting of a control unit 30 that is battery operated or ma be powered by the means of the power input 32. A swing out USB connection 34 optionally located on the control unit may be directly connected to a computer. The control unit 30 will have a recording output line 38 going out to the inner sound and motion activation unit 42. One or more speakers 48 are conveniently placed in the area of the baby's head. One or more locating square orifices 50 are centrally located to match the square locating hook-loop fastening squares 52 centrally located on either side within the outer enclosure 14 holding the inner sound and motion activation unit 42 firmly in position.

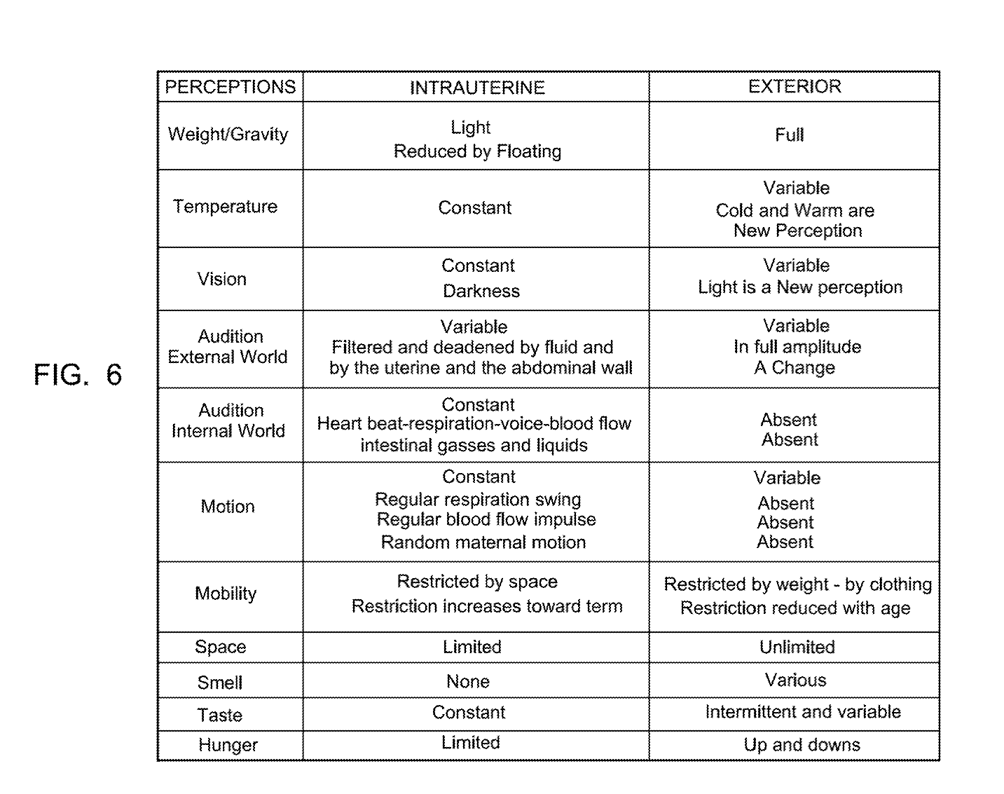

FIG. 6 depicts a chart showing a baby's intrauterine and after birth exterior perceptions. Audition and motion are the key components for the present invention.

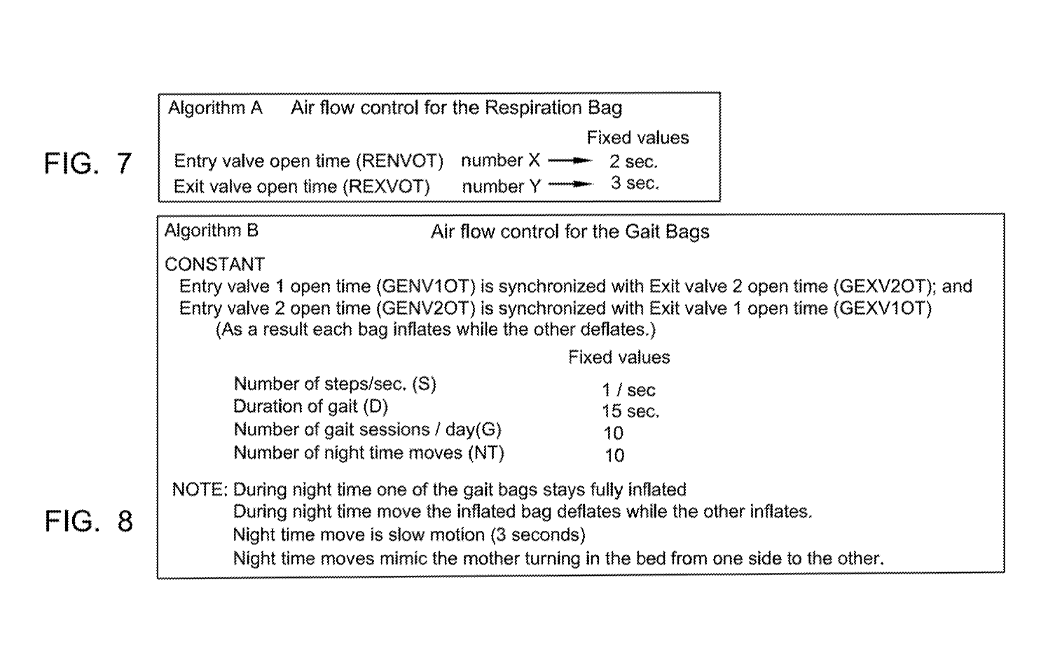

FIG. 7 depicts algorithm A depicting the air flow control for the respiration bag with fixed values. The term RENVOT stands for the respiration simulation entry valve open time, and the term REXVOT stands for the respiration simulation exit valve open time.

FIG. 8 depicts algorithm B depicting the air flow control for the gait bag. The term GENV1OT stands for the gait simulation entry valve 1 open time. GENV2OT stands for the gait simulation entry valve 2 open time, GEXV1OT stands for the gait simulation exit valve 1 open time, and GEXV2OT stands for the gait simulation exit valve 2 open time.

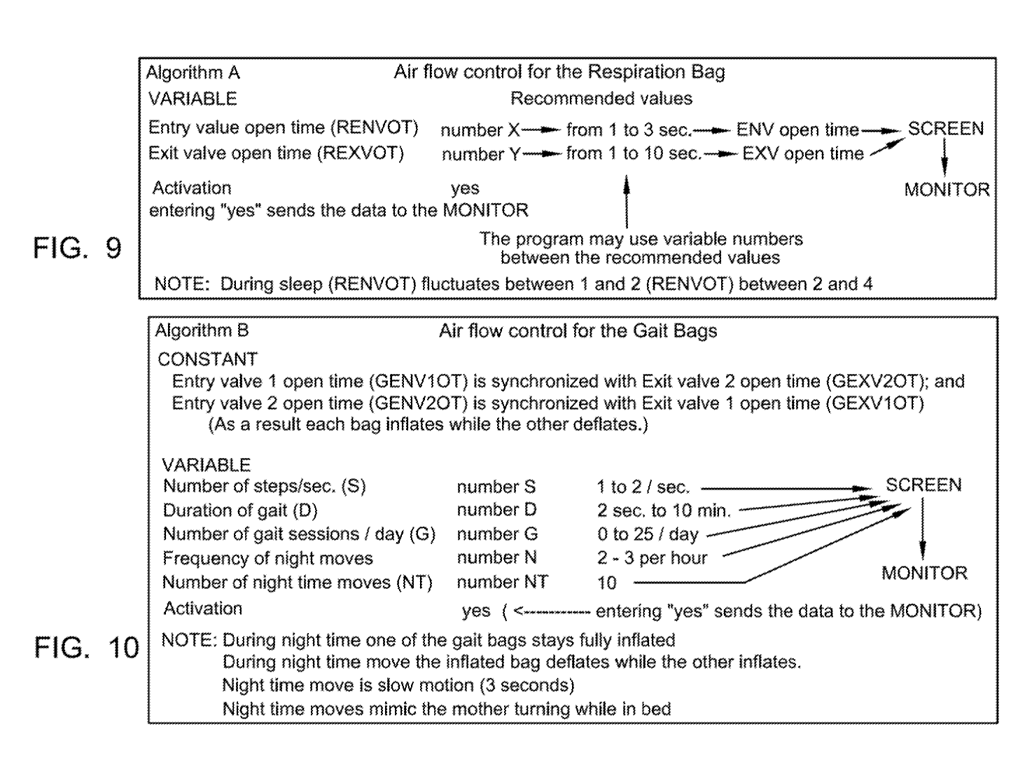

FIG. 9 depicts algorithm A depicting the air flow control for the respiration bag. The term RENVOT stands for the respiration simulation entry valve open time, and the term REXVOT stands for the respiration simulation exit valve open time.

FIG. 10 depicts algorithm B depicting the air flow for the gait bag. The term GENV1OT stands for the gait simulation entry valve 1 open time, GENV2OT stands for the gait simulation entry valve 2 open time. GEXV1OT stands for the gait simulation exit valve 1 open time, and GEXV2OT stands for the gait simulation exit valve 2 open time.

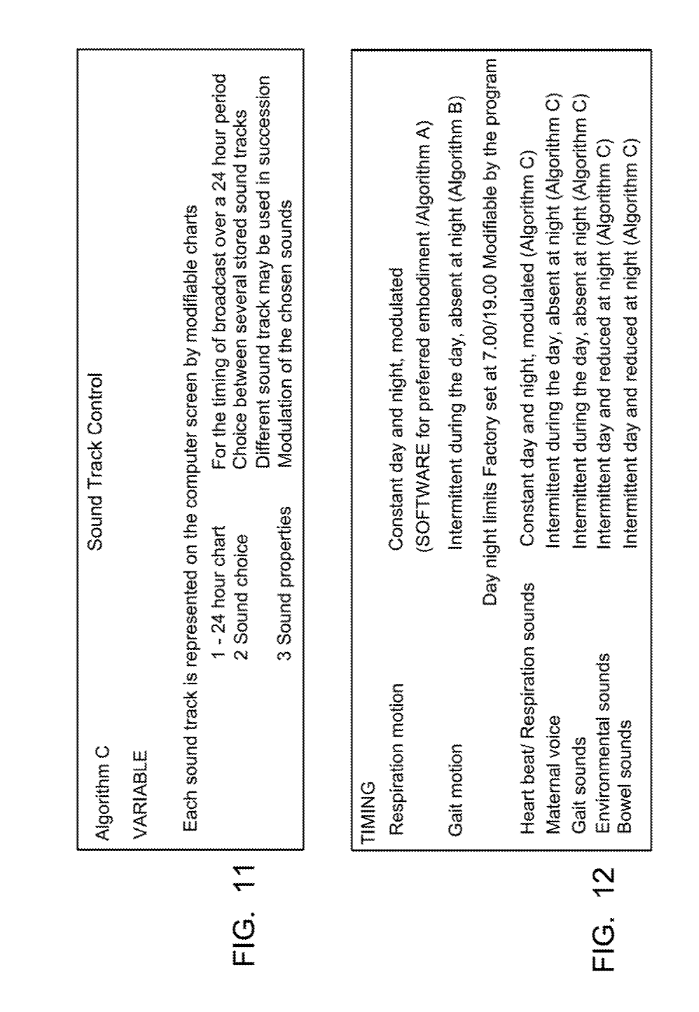

FIG. 11 depicts algorithm C depicting the sound track control. The sound track may be programmed and played back in a variety of sequences facilitating clinical research into the effects of varying sound on the prematurely born or newborn baby.

FIG. 12 depicts a timing chart for respiration and gait motion, as well as heart beat, respiration, maternal voice, gait, bowel and environmental sounds.

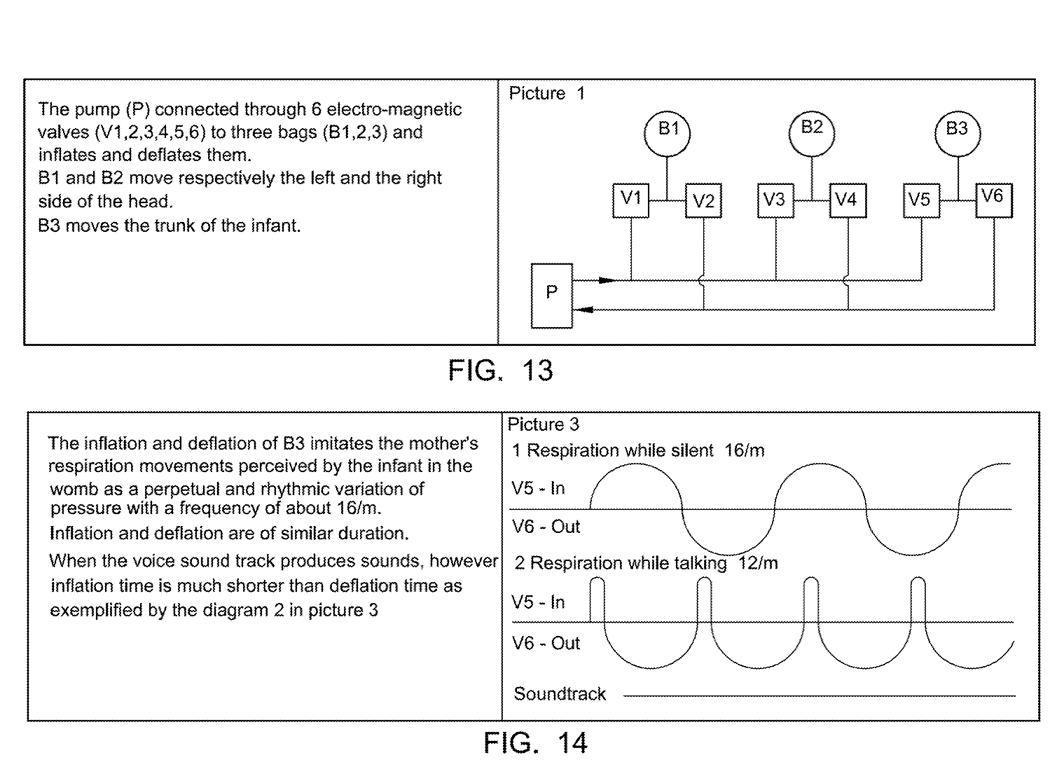

FIG. 13 depicts a chart depicting the pump connections for the respiration bags. The pump is connected through 6 electro-magnetic valves to three bags and inflates and deflates them. B1 and B2 move respectively, the left and right side of the head while B3 moves the trunk of the infant.

FIG. 14 depicts a chart depicting the pump sequences for the respiration bags. The inflation and deflation of B3 imitates the mother's respiration movements perceived by the infant in the womb as a perpetual and rhythmic variation of pressure with a frequency of about 16 per minute. When the voice sound track produces voice sounds, inflation time is much shorter that deflation time as shown in the chart.

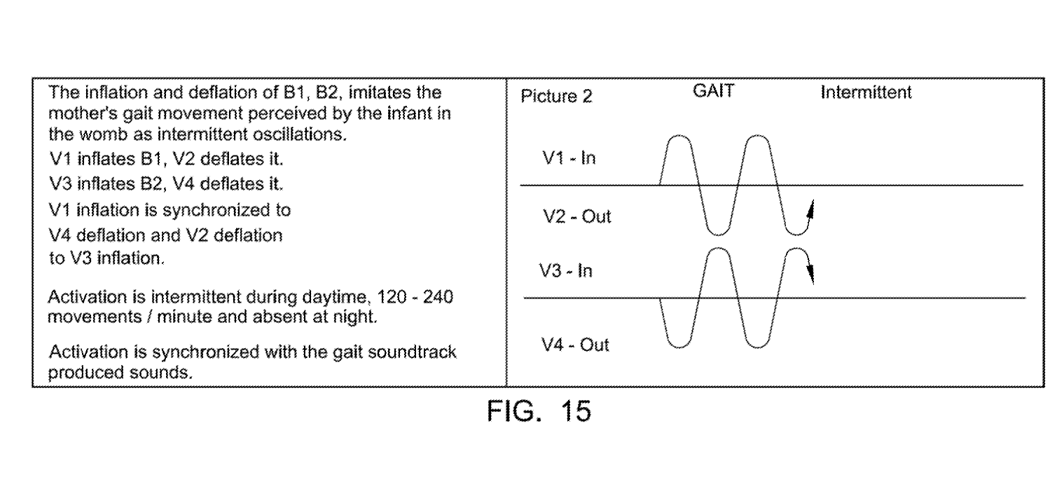

FIG. 15 depicts a chart depicting the gait valve activation. The inflation and deflation of B1 and B2 imitates the mother's gait movement perceived by the infant in the womb as intermittent oscillations. Activation varies during night and day time periods. Activation is intermittent during the day, at about 120 to about 240 movements per minute and absent at night. Activation is synchronized with the gait soundtrack produced sounds.

Table 1 seen below explains the various features found in the four anticipated example product models. Example Product Model 1 consists of sound capabilities only. This model can be incorporated into infant products such as teddy bears and the like, as well as being in the form of a newborn comfort mat. Example Product Model 2 will have both sound and motion capabilities. Example Product Model 3 adds a controller capable of connecting to a computer, to control sound tracks as well as an air/vacuum pump for facilitating the sound and motion. Example Product Model 4 is the same as model 3, except that the source of air/vacuum will be the hospital connections in the wall leading to a central hospital air supply and vacuum pull. The various examples shown in the above described figures are one or more of the outlined models as exemplified in Table 1. Many other models are anticipated, including those equipped with recorders for recording both sound and motion.

TABLE 1
EXAMPLE PRODUCT MODELS

| | EXAMPLE PRODUCT MODELS | | | |
|---|---|---|---|---|
| | 1 | 2 | 3 | 4 |
| INNER UNITS | | | | |
| Speakers | YES | YES | YES | YES |
| Motion bags | NO | YES | YES | YES |
| CONTROLER | | | | |
| Programmable | NO | NO | YES | YES |
| RECORDER | NO | NO | YES | YES |
| AIR/VACUUM PUMPS | NO | YES | YES | NO |
| HOSPITAL IN WALL | NO | NO | NO | YES |
| AIR/VACUUM SOURCE | | | | |

The Uterine Sound and Motion Simulation Devices 10A, 10B and 10C shown in the drawings and described in detail herein disclose arrangements of elements of particular construction and configuration for illustrating preferred embodiments of structure and method of operation of the present application. It is to be understood, however, that elements of different construction and configuration and other arrangements thereof, other than those illustrated and described may be employed for providing a Uterine Sound and Motion Simulation Device 10A, 10B and 10C in accordance with the spirit of this disclosure, and such changes, alternations and modifications as would occur to those skilled in the art are considered to be within the scope of this design as broadly defined in the appended claims.

Further, the purpose of the foregoing abstract is to enable the U.S. Patent and Trademark Office and the public generally, and especially the scientists, engineers and practitioners in the art who are not familiar with patent or legal terms or phraseology, to determine quickly from a cursory inspection the nature and essence of the technical disclosure of the application. The abstract is neither intended to define the invention of the application, which is measured by the claims, nor is it intended to be limiting, as to the scope of the invention in any way.

### INDUSTRIAL APPLICABILITY

The present invention may be used in all of its varying configurations and models in the care of newborn or prematurely born babies in the neonatal intensive care units of hospitals to help alleviate the consequences of solitary confinement of premature babies in their incubator on later development and behavior and as used in the home environment the present invention will be useful in helping to alleviate the consequences of endless crying on the development of the newborn. Additionally, the present invention can be used for clinical research into the effects of varying sound and motion on the prematurely horn or newborn infant.

I claim:

1. A uterine sound and motion simulation device comprising:
(a) an inner sound and motion activation unit forming a flat mat;
(b) motion bladders and motion pillows embedded into said activation unit;
(c) sound emitting speakers embedded into said activation unit;
(d) a control unit capable of controlling the activation of said motion bladders and pillows and said sound emitting speakers; and
(e) an outer enclosure removeably enclosing said activation unit.

2. The uterine sound and motion simulation device, according to claim 1, wherein said motion bladders and pillows are activated by an air/vacuum source and said air/vacuum source is controlled by said control unit which is in direct communication with said air/vacuum source.

3. The uterine sound and motion simulation device, according to claim 1, wherein said sound emitting speakers are directly controlled by said control unit which is in direct communication with said speakers.

4. The uterine sound and motion simulation device, according to claim 1, wherein said control unit is capable of connection to a central processing unit (CPU), and further wherein said CPU is employed to program varying sequences of motion and sound.

5. The uterine sound and motion simulation device, according to claim 2, further comprising a plurality of valves, wherein said control unit is in direct communication with said plurality of valves which direct and control the air and vacuum coming from said air/vacuum source.

6. The uterine sound and motion simulation device, according to claim 5, wherein said air/vacuum source is a stand-alone air/vacuum pump unit.

7. The uterine sound and motion simulation device, according to claim 5, wherein said air/vacuum source is a central hospital wall outlet air source connection and a central hospital wall vacuum source connection.

8. The uterine sound and motion simulation device, according to claim 1, wherein said control unit is powered by an AC power source.

9. The uterine sound and motion simulation device, according to claim 1, wherein said control unit is powered by a battery power source.

10. The uterine sound and motion simulation device, according to claim 1, wherein said outer enclosure is in the form of a washable baby blanket.

11. The uterine sound and motion simulation device, according to claim 1, further including a recording device having a plurality of microphones connected to said recorder device via a plurality of wires, wherein said microphones are positioned on the body of a pregnant woman or new mother for the purpose of recording inner and outer sounds.

12. The uterine sound and motion simulation device, according to claim 11, wherein said recorder device is capable of recording motion as well as sound.

13. A method for making a uterine sound and motion simulation device, comprising the steps of:
(a) providing an inner sound and motion activation unit forming a flat mat;
(b) providing motion bladders and motion pillows embedded into said activation unit;
(c) providing sound emitting speakers embedded into said activation unit;
(d) providing a control unit capable of controlling the activation of said motion bladders and pillows and said sound emitting speakers; and
(e) providing an outer enclosure removeably enclosing said activation unit.

14. The method of making a uterine sound and motion simulation device, according to claim 13, wherein said step of providing motion bladders and motion pillows embedded into said activation unit, further includes providing motion bladders and motion pillows embedded into said activation unit wherein said motion bladders and pillows are activated by an air/vacuum source and said air/vacuum source is controlled by said control unit which is in direct communication with said air/vacuum source.

15. The method of making a uterine sound and motion simulation device, according to claim 13, wherein said step of providing sound emitting speakers embedded into said activation unit further includes providing sound emitting speakers embedded into said activation unit wherein said sound emitting speakers are directly controlled by said control unit which is in direct communication with said speakers.

16. The method of making a uterine sound and motion simulation device, according to claim 13, wherein said step of providing a control unit capable of controlling the activation of said motion bladders and pillows and said sound emitting speakers, further includes providing a control unit capable of controlling the activation of said motion bladders and pillows and said sound emitting speakers wherein said control unit is capable of connection to a computer central processing unit, and further wherein said computer central processing unit is employed to program varying sequences of motion and sound.

17. The method of making a uterine sound and motion simulation device, according to claim 13, further including the step of providing a plurality of valves and further wherein said control unit is in direct communication with said plurality of valves which direct and control the air and vacuum coming from said air/vacuum source and going to said provided motion bladders and motion pillows thereby actuating said motion bladders and motion pillows by filling with air and evacuating that air.

18. The method of making a uterine sound and motion simulation device, according to claim 17, wherein said step of providing a plurality of valves in direct communication with said control unit further includes the step of providing an air/vacuum source wherein said air/vacuum source is a stand-alone air/vacuum pump unit.

19. The method of making a uterine sound and motion simulation device, according to claim 17, wherein said step of providing a plurality of valves in direct communication with said control unit further includes the step of providing an air/vacuum source wherein said air/vacuum source is a central hospital wall outlet air source connection and a central hospital wall vacuum source connection.

20. The method of making a uterine sound and motion simulation device, according to claim 13, wherein said step of providing a control unit capable of controlling the activation of said motion bladders and pillows and said sound emitting speakers, further includes the step of providing a control unit capable of controlling the activation of said motion bladders and pillows and said sound emitting speakers wherein said control unit is powered by an AC power source.

21. The method of making a uterine sound and motion simulation device, according to claim 13, wherein said step of providing a control unit capable of controlling the activation of said motion bladders and pillows and said sound emitting speakers, further includes the step of providing a control unit capable of controlling the activation of said motion bladders and pillows and said sound emitting speakers wherein said control unit is powered by a battery power source.

22. The method of making a uterine sound and motion simulation device, according to claim 13, wherein said step of providing an outer enclosure removeably enclosing said activation unit, further includes the step of providing an outer enclosure removeably enclosing said activation unit wherein said outer enclosure is in the form of a washable baby blanket.

23. The method of making a uterine sound and motion simulation device, according to claim 13, further including the step of providing a recording device having a plurality of microphones connected to said recorder device via a plurality of wires, wherein said microphones are positioned on the body of a pregnant woman or new mother for the purpose of recording inner and outer sounds.

24. The method of making uterine sound and motion simulation device, according to claim 23, wherein said step of providing a recorder device, further includes the step of providing a recorder device which is capable of recording motion as well as sound.
